(19) United States
(12) Patent Application Publication
(10) Pub. No.: US 2007/0155987 A1
(43) Pub. Date: Jul. 5, 2007

(54) Oxidative digestion with optimized agitation

(76) Inventors: Ruairi Seosamh O'Meadhra, Kingsport, TN (US); Robert Lin, Kingsport, TN (US)

Correspondence Address: Steven A. Owen;Eastman Chemical Company, P.O. Box 511, Kingsport, TN, 37662-5075, US

(21) Appl. No.: 11/325,295

(22) Filed: Jan. 4, 2006

(51) Int. Cl. C07C 51/42

(52) U.S. Cl. 562/485

(57) ABSTRACT

An apparatus and process for purifying crude particles of an aromatic dicarboxylic acid (e.g., crude terephthalic acid) via oxidative digestion in an agitated reactor. Purification and particle size of the particles exiting the digestion reactor are optimized by controlling the amount of mechanical agitation imparted to the reaction medium in the digestion reactor.

### FIELD OF THE INVENTION

This invention relates generally to the production and purification of acids, such as aromatic dicarboxylic acids. In another aspect, the invention concerns an improved method for purifying a slurry containing solid particles of crude terephthalic acid (CTA).

### BACKGROUND OF THE INVENTION

Liquid-phase oxidation reactions are employed in a variety of commercial processes. A particularly significant commercial oxidation process is the liquid-phase catalytic partial oxidation of para-xylene to terephthalic acid. Terephthalic acid is an important compound with a variety of applications. The primary use of terephthalic acid is as a feedstock in the production of polyethylene terephthalate (PET). PET is a well-known plastic used in great quantities around the world to make products such as bottles, fibers, and packaging.

In a typical liquid-phase oxidation process, a liquid-phase feed stream and a gas-phase oxidant stream are introduced into a reactor and form a multi-phase reaction medium therein. In the production of terephthalic acid, the liquid-phase feed stream typically contains para-xylene and the gas-phase oxidant stream contains molecular oxygen. At least a portion of the molecular oxygen introduced into the reactor as a gas dissolves into the liquid phase of the reaction medium to make oxygen available for the liquid-phase reaction.

The product withdrawn from the main oxidizer of conventional terephthalic acid production processes is typically a slurry containing particles of crude terephthalic acid (CTA) and a mother liquor. CTA contains relatively high levels of impurities (e.g., 4-carboxybenzaldehyde, para-toluic acid, fluorenones, and other color bodies) that render it unsuitable as a feedstock for the production of PET. Thus, the CTA produced in conventional oxidation processes must be subjected to a purification process that converts the CTA into a purified terephthalic acid (PTA) suitable for making PET.

One process for converting CTA to PTA involves subjecting the CTA particles to oxidative digestion. Oxidative digestion is typically carried out in a mechanically-agitated reactor. During oxidative digestion, the CTA particles produced in the primary oxidizer are partially or fully dissolved in the liquid phase of the reaction medium in the digestion reactor. This dissolution allows impurities trapped within the CTA particles to be released into the liquid phase where they can be subjected to liquid-phase oxidation. During oxidative digestion, particles containing terephthalic acid are continuously dissolving and reprecipitating. The reprecipitated particles have a reduced impurities content as compared to the CTA particles introduced in to the digestion reactor.

### OBJECTS AND SUMMARY OF THE INVENTION

It has been discovered that the impurity-reducing effectiveness of oxidative digestion can be greatly influenced by the amount of agitation imparted to the reaction medium contained in the digestion reactor. Further, the particle size of the PTA particles withdrawn from of the oxidative digestion reaction can be greatly influenced by the amount of agitation imparted to the reaction medium in the digestion reactor.

Accordingly, it is an object of the present invention to provide a process and apparatus for carrying out oxidative digestion under optimized agitation conditions.

In accordance with one embodiment of the present invention there is provided a method of purifying a crude slurry comprising particles of crude terephthalic acid (CTA). The method comprises: (a) introducing the crude slurry into a digestion reactor containing a multi-phase reaction medium, wherein the digestion reactor employs at least one mechanical stirrer having less than five impellers to agitate the reaction medium; and (b) reacting at least a portion of the multi-phase reaction medium in the digestion reactor, wherein during reacting the ratio of the amount of power consumed by the mechanical stirrer to the volume of the reaction medium is in the range of from about 0.05 to about 1.5 kw/m³.

In accordance with another embodiment of the present invention there is provided a method of making terephthalic acid (TPA). The method comprises: (a) oxidizing an aromatic compound in a primary oxidation reactor to thereby produce a crude slurry comprising crude terephthalic acid (CTA) particles; and (b) subjecting at least a portion of the CTA particles to oxidation in a digestion reactor to thereby produce a slurry comprising purer terephthalic acid (PTA) particles, wherein the digestion reactor includes a mechanical stirrer having less than five impellers, wherein during the oxidation in the digestion reactor the ratio of the amount of power consumed by the mechanical stirrer to the internal volume of the digestion reactor is in the range of from about 0.05 to about 1.5 kw/m³.

In accordance with still another embodiment of the present invention there is provided an apparatus comprising a primary oxidation vessel, a digestion vessel in fluid communication with the primary oxidation vessel; and a mechanical stirrer at least partly disposed in the digestion vessel and having less than five impellers. The mechanical stirrer is configured to consume power at a rate in the range of from about 0.05 to about 1.5 kilowatts per cubic meter of volume defined within the digestion vessel.

### BRIEF DESCRIPTION OF THE DRAWINGS

FIG. 1 is a sectional side view of an agitated digestion reactor constructed in accordance with one embodiment of the present invention.

FIG. 2 is a schematic diagram of a process for producing terephthalic acid employing a digestion reactor constructed and operated in accordance with the present invention.

### DETAILED DESCRIPTION OF THE INVENTION

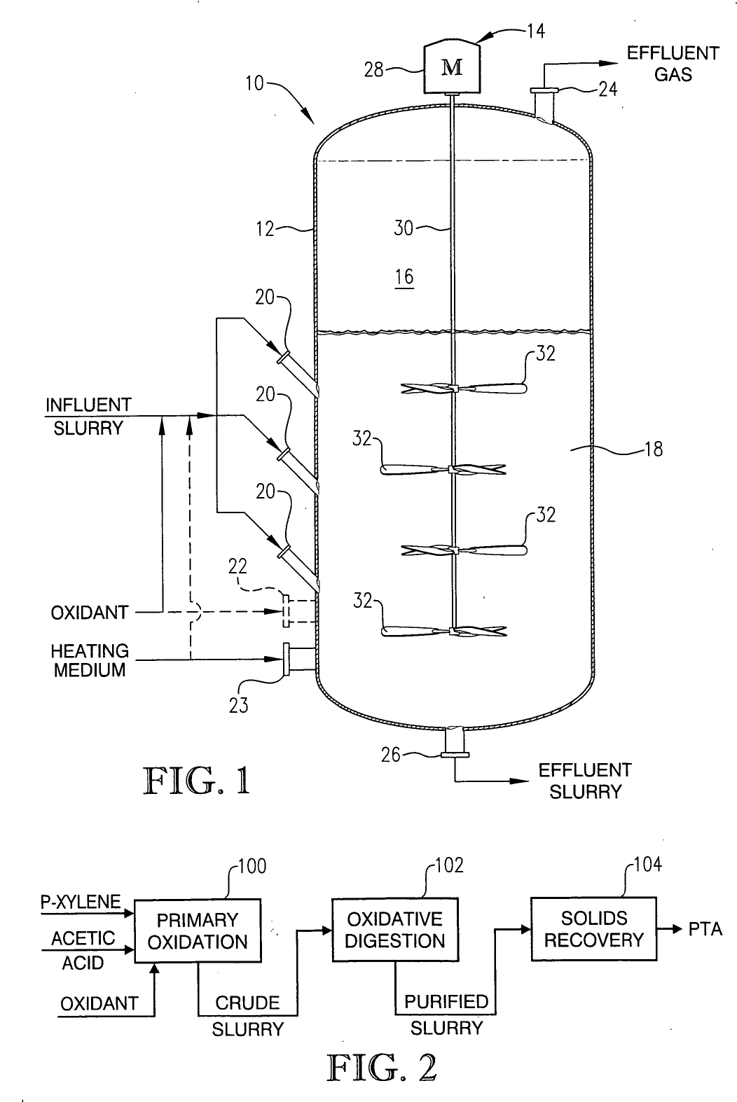

Referring initially to FIG. 1, a mechanically-agitated oxidative digestion reactor 10 is illustrated as generally comprising a vessel shell 12 and a mechanical agitation system 14. Vessel shell 12 defines a reaction zone 16 within which a multi-phase reaction medium 18 is contained. Digestion reactor 10 includes one or more slurry inlets 20a,b,c for receiving an influent slurry into reaction zone 16. Digestion reactor 10 can optionally be equipped with a separate oxidant inlet 22 for receiving an oxidant stream into reaction zone 16. Digestion reactor 10 can also be equipped with a heating inlet 23 for receiving a heating medium into reaction zone 16. A gas outlet 24 is preferably located near the top of digestion reactor 10, while a slurry outlet 26 is preferably located near the bottom of reactor 10. An effluent gas is discharged from reaction zone 16 via gas outlet 24, while an effluent slurry is discharged from reaction zone 16 via slurry outlet 26.

Vessel shell 12 preferably has a generally upright orientation so that the height of reaction zone 16 is greater than the width of reaction zone 16. Vessel shell 12 is preferably configured such that the maximum height (H) of reaction zone 16 is in the range of from about 5 to about 50 meters, more preferably in the range of from about 8 to about 25 meters, and most preferably in the range of from 10 to 15 meters. Preferably, the maximum width (W) of reaction zone 16 is in the range of from about 1 to about 20 meters, more preferably in the range of from about 2 to about 10 meters, and most preferably in the range of from 3 to 6 meters. Preferably, the H/W ratio of reaction zone 16 is at least about 0.5, more preferably at least about 1, and most preferably in the range of from 1.5 to 5. The total volume of reaction zone 16 is preferably at least about 50 cubic meters (m³), more preferably in the range of from about 100 to about 5,000 m³, and most preferably in the range of from about 500 to about 2,000 m³.

Mechanical agitation system 14 preferably includes a rotational driver 28, a shaft 30, and a plurality of spaced-apart impellers 32. In a preferred embodiment of the present invention, rotational driver 28 is an electric motor disposed at or near the top of vessel shell 12 and shaft 30 has a generally upright orientation. Most preferably, shaft 30 has a substantially vertical orientation. Driver 28 is coupled to shaft 30 in a manner that permits driver 28 to rotate shaft 30. Rotation of shaft 30 causes rotation of impellers 32. During normal operation, impellers 32 are submerged in reaction medium 18 so that rotation of impellers 32 causes agitation of reaction medium 18. Preferably, reaction medium 18 fills in the range of from about 50 to about 100 percent of the volume of reaction zone 16, most preferably in the range of from 60 to 80 percent of the volume of reaction zone 16.

In an alternative embodiment of the present invention, mechanical agitation system 14 includes more than one driver 28 and/or shaft 30. In one embodiment of the present invention, it is preferred for mechanical agitation system 14 to include less than 5 impellers per shaft, more preferably 2 to 4 impellers per shaft. In another embodiment, mechanical agitation system includes 2 to 10 impellers per shaft, more preferably 3 to 8 impellers per shaft, and most preferably 4 to 6 impellers per shaft.

In a preferred embodiment of the present invention, mechanical agitation system 14 is configured so that the total power consumed by mechanical agitation system 14 during steady-state operation of digestion reaction 10 is in the range of from about 0.05 to about 1.5 kilowatts per cubic meter of reaction medium 18 or reaction zone 16 (kw/m³), more preferably in the range of from about 0.1 to about 0.9 kw/m³, and most preferably in the range of from 0.2 to 0.8 kw/m³. Preferably, the total power consumed by mechanical agitation system 14 per impeller is in the range of from about 0.01 to about 0.3 kw/m³, more preferably in the range of from about 0.02 to about 0.18 kw/m³, and most preferably in the range of from 0.04 to 0.16 kw/m³. During steady-state operation of digestion reactor 10 it is preferred for the average rotational speed of impellers 32 to be maintained in the range of from about 20 to about 120 revolutions per minute (rpm), most preferably in the range of from 30 to 90 rpm. The total volume of reaction medium 18 in digestion reactor 10 is preferably at least about 50 m³, more preferably in the range of from about 100 to about 5,000 m³, and most preferably in the range of from about 500 to about 2,000 m³.

Referring again to FIG. 1, during normal operation of digestion reactor 10, an influent slurry is introduced into reaction zone 16 via one or more slurry inlets 20. The influent slurry comprises solid particles having one or more impurities trapped therein. The influent slurry preferably comprises at least about 10 weight percent solids, more preferably in the range of from about 20 to about 40 weight percent solids, and most preferably in the range of from 25 to 35 weight percent solids.

In a preferred embodiment of the present invention, the majority (i.e., >50 wt. %) of the solid particles contained in the influent slurry are solid particles of crude terephthalic acid (CTA) that contain impurities such as 4-carboxybenzaldehyde (4-CBA) and para-toluic acid (P-TAc). These CTA particles preferably contain at least about 400 parts per million by weight (ppmw) of 4-CBA, more preferably at least about 800 ppmw of 4-CBA, and most preferably in the range of from about 1,000 to 15,000 ppmw of 4-CBA.

In digestion reactor 10, reaction medium 18 is subjected to liquid phase oxidation so as to oxidize at least a portion of the impurities present in the influent slurry. In order to facilitate oxidation in digestion reactor 10, an oxidant stream is added upstream of and/or directly into digestion reactor 10. The oxidant stream can be any stream capable of providing and/or generating a sufficient amount of oxygen in reaction medium 18 to facilitate liquid-phase oxidation of at least a portion of the impurities contained in the influent slurry. Preferably, the oxidant stream comprises in the range of from about 5 to about 40 mole percent molecular oxygen, more preferably in the range of from about 15 to about 30 mole percent molecular oxygen, and most preferably in the range of from 18 to 24 mole percent molecular oxygen. It is preferred for the balance of the oxidant stream to be comprised primarily of a gas or gasses, such as nitrogen, that are inert to oxidation. More preferably, the oxidant stream consists essentially of molecular oxygen and nitrogen. Most preferably, the oxidant stream is dry air that comprises about 21 mole percent molecular oxygen and about 78 to about 81 mole percent nitrogen.

In order to facilitate oxidation of impurities in digestion reactor 10, it is preferred for reaction medium 18 to be maintained at a temperature in the range of from about 165 to about 230° C., more preferably in the range of about 175 to about 220° C., most preferably in the range of from 185 to 210° C. and a pressure in the range of from about 1 to about 20 bar, more preferably in the range of from about 2 to about 12 bar, and most preferably in the range of from 4 to 8 bar. The temperature of reaction medium 18 can be controlled by the addition of a heating medium via heating inlet 24 and/or via combination with the influent slurry upstream of slurry inlets 20. In a preferred embodiment, the heating medium comprises acetic acid. Most preferably, the heating medium is a vapor that contains at least 75 mole percent acetic acid.

During processing in digestion reactor 10, the solid particles in reaction medium 18 dissolve and reprecipitate. This dissolution and reprecipitation in reaction medium 18 allows impurities originally trapped in the solid particles of the influent slurry to enter the liquid phase of reaction medium 18, where the impurities can be subjected to liquid-phase oxidation. Although the solid particles in reaction medium 18 are constantly dissolving and reprecipitating, it is preferred for the average solids content of reaction medium 18 to be at least about 5 weight percent, more preferably in the range of from about 10 to about 60 weight percent, and most preferably in the range of from 15 to 40 weight percent.

The effluent slurry withdrawn from reaction zone 16 via slurry outlet preferably comprises at least about 5 weight percent solids, more preferably in the range of from about 15 to about 35 weight percent solids, and most preferably in the range of from 20 to 30 weight percent solids. When the influent slurry to reaction zone 16 contains solid particles of CTA, it is preferred for the effluent slurry withdrawn from reaction zone 16 to contain solid particles of purer terephthalic acid (PTA). These PTA particles preferably contain at least about 100 ppmw less 4-CBA than the original CTA particles, more preferably at least about 200 ppmw less 4-CBA, and most preferably at least 400 ppmw less 4-CBA. Preferably, the PTA in the effluent slurry comprises less than about 400 ppmw of 4-CBA, more preferably less than about 250 ppmw of 4-CBA, and most preferably in the range of from 10 to 200 ppmw of 4-CBA.

FIG. 2 illustrates an improved process for producing PTA employing a primary oxidation reactor 100, an oxidative digestion reactor 102, and a solids recovery system 104. Digestion reactor 102 is preferably configured in accordance with an embodiment of the present invention.

Referring again to FIG. 2, in a preferred embodiment, one or more streams containing para-xylene, acetic acid, and an oxidant are charged to primary oxidation reactor 100. A multi-phase reaction medium is formed in primary oxidation reactor 100, and partial liquid-phase oxidation of para-xylene to terephthalic acid is carried out therein. A catalyst system facilitates the liquid-phase oxidation in reactor 100. Preferably, the catalyst system comprises at least one multivalent transition metal. More preferably, the multivalent transition metal comprises cobalt. Even more preferably, the catalyst system comprises cobalt and bromine. Most preferably, the catalyst system comprises cobalt, bromine, and manganese.

During oxidation in primary oxidation reactor 100, it is preferred for the reaction medium contained therein to be maintained at a temperature in the range of from about 125 to about 200° C., more preferably in the range of from about 140 to about 180° C., and most preferably in the range of from 150 to 170° C. The overhead pressure above reaction medium 36 is preferably maintained in the range of from about 1 to about 20 bar gauge (barg), more preferably in the range of from about 2 to about 12 barg, and most preferably in the range of from 4 to 8 barg.

In a preferred embodiment of the present invention, primary oxidation reactor 100 is configured and operated in a manner that produces a crude slurry containing CTA particles that are particularly well suited for purification by oxidative digestion. In a preferred embodiment of the present invention primary oxidation reactor 100 is a bubble column reactor configured and operated in the manner described in U.S. patent application Ser. No. 11/154,219, the entire disclosure of which is incorporated by reference herein to the extent that it does not conflict with the description of the present invention.

Preferably, a substantial portion of the CTA particles produced by primary oxidation reactor 100 are each formed of a plurality of small, agglomerated CTA subparticles, thereby giving the base CTA particles a relatively high surface area, high porosity, low density, and good dissolvability. The base/agglomerated CTA particles preferably have a mean particle size in the range of from about 20 to about 150 microns, more preferably in the range of from about 30 to about 120 microns, and most preferably in the range of from 40 to 90 microns. The CTA subparticles that agglomerate to form the base CTA particles preferably have a mean particle size in the range of from about 0.5 to about 30 microns, more preferably from about 1 to about 15 microns, and most preferably in the range of from 2 to 5 microns.

The relatively high surface area of the base CTA particles produced in primary oxidation reactor 100, can be quantified using a Braunauer-Emmett-Teller (BET) surface area measurement method. Preferably, the base CTA particles have an average BET surface of at least about 0.6 meters squared per gram (m²/g). More preferably, the base CTA particles have an average BET surface area in the range of from about 0.8 to about 4 m²/g. Most preferably, the base CTA particles have an average BET surface area in the range of from 0.9 to 2 m²/g.

The crude slurry withdrawn from primary oxidation reactor 100 can be employed directly as the influent slurry to digestion reactor 102. Alternatively, a portion of the liquid mother liquor exiting primary oxidation reactor 102 can be replaced with clean liquor prior to introduction into digestion reactor 102. In digestion reactor 102, the slurry is processed in accordance with the description provided above with respect to FIG. 1. Preferably, the temperature of oxidative digestion in digestion reactor 102 is at least 20° C. greater than the temperature of oxidation in primary oxidation reactor 100. More preferably, the temperature of oxidative digestion in digestion reactor 102 is in the range of from 25 to 50° C. greater than the temperature of oxidation in primary oxidation reactor 100.

The configuration and operation of digestion reactor 102 is optimized to control the size of the purer terephthalic acid (PTA) particles of the purified slurry produced therefrom. Preferably, the PTA particles exiting digestion reactor 102 have a mean particle size that is at least about 20 percent smaller than the mean particle size of the base CTA particles entering digestion reactor 102. Most preferably, the PTA particles exiting digestion reactor 102 have a mean particle that is at least 50 percent smaller than the mean particle size of the base CTA particles entering digestion reactor 102. Preferably, the PTA particles exiting digestion reactor 102 have a mean particle in the range of from about 30 to about 100 microns, most preferably in the range of from 40 to 80 microns.

The size of the PTA particles exiting digestion reactor 102 make them well suited for recovery in solids recovery system 104. Solids recovery system 104 comprises one or more items of equipment known in the art for recovering solids from a slurry.

The inventors note that for all numerical ranges provided herein, the upper and lower ends of the ranges can be independent of one another. For example, a numerical range of 10 to 100 means greater than 10 and/or less than 100. Thus, a range of 10 to 100 provides support for a claim limitation of greater than 10 (without the upper bound), a claim limitation of less than 100 (without the lower bound), as well as the full 10 to 100 range (with both upper and lower bounds).

The invention has been described in detail with particular reference to preferred embodiments thereof, but will be understood that variations and modification can be effected within the spirit and scope of the invention.

That which is claimed is:

1. A method of purifying a crude slurry comprising particles of crude terephthalic acid (CTA), said method comprising: (a) introducing said crude slurry into a digestion reactor containing a multi-phase reaction medium, wherein said digestion reactor employs at least one mechanical stirrer having less than five impellers to agitate said reaction medium; and (b) reacting at least a portion of said multi-phase reaction medium in said digestion reactor, wherein during said reacting the ratio of the amount of power consumed by said mechanical stirrer to the volume of said reaction medium is in the range of from about 0.05 to about 1.5 kw/m³.

2. The method of claim 1 wherein the ratio of the amount of power consumed by said mechanical stirrer to the volume of said reaction medium is in the range of from about 0.1 to about 0.9 kw/m³.

3. The method of claim 1 wherein said reacting includes oxidizing.

4. The method of claim 1 wherein said reacting takes place in a liquid phase of said multi-phase reaction medium.

5. The method of claim 1 wherein said crude slurry has a solids content of at least about 10 percent by weight.

6. The method of claim 1 further comprising, introducing molecular oxygen into said crude slurry and/or said reaction medium.

7. The method of claim 1 further comprising, introducing vapors into said reaction medium.

8. The method of claim 7 wherein said vapors heat said reaction medium.

9. The method of claim 8 wherein said vapors comprise acetic acid.

10. The method of claim 1 wherein said multi-phase reaction medium is maintained at a temperature in the range of from about 165 to about 23° C.

11. The method of claim 1 wherein said reaction medium has a volume of at least about 50 m³.

12. The method of claim 1 wherein the rotational speed of said mechanical stirrer is maintained in the range of from about 20 to about 120 revolutions per minute (rpm).

13. The method of claim 1 further comprising, withdrawing at least a portion of said reaction medium from said digestion reactor as a purified slurry comprising particles of purer terephthalic acid (PTA), wherein the 4-carboxy benzaldehyde (4-CBA) content of said PTA particles is less than the 4-CBA content of said CTA particles.

14. The method of claim 13 wherein said crude slurry has a solids content in the range of from about 20 to about 40 percent by weight, wherein said purified slurry has a solids content in the range of from about 15 to about 35 percent by weight.

15. The method of claim 13 wherein said crude slurry comprises agglomerations of said CTA particles, wherein said agglomerations have a mean particles size in the range of from about 50 to about 150 microns.

16. The method of claim 15 wherein the mean particle size of said PTA particles is at least about 20 microns less than the mean particle size of said crude agglomerations.

17. The method of claim 13 wherein the mean particle size of said PTA particles is in the range of from about 30 to about 100 microns.

18. The method of claim 13 further comprising, dissolving at least a portion of said CTA particles in said reaction medium and precipitating at least a portion of said PTA particles in said reaction medium.

19. A method of making terephthalic acid (TPA), said method comprising: (a) oxidizing an aromatic compound in a primary oxidation reactor to thereby produce a crude slurry comprising crude terephthalic acid (CTA) particles; and (b) subjecting at least a portion of said CTA particles to oxidation in a digestion reactor to thereby produce a slurry comprising purer terephthalic acid (PTA) particles, wherein said digestion reactor includes a mechanical stirrer having less than five impellers, wherein during said oxidation the ratio of the amount of power consumed by said mechanical stirrer to the internal volume of said digestion reactor is in the range of from about 0.05 to about 1.5 kw/m³.

20. The method of claim 19 wherein said oxidation in said digestion reactor is carried out at a temperature that is at least about 20° C. greater than the temperature at which said oxidizing in said primary oxidation reactor is carried out.

21. The method of claim 20 wherein said oxidizing in said primary oxidation reactor is carried out at a temperature in the range of from about 120 to about 200° C., wherein said oxidizing in said oxidation in said digestion reactor is carried out at a temperature in the range of from about 165 to about 230° C.

22. The method of claim 19 wherein said aromatic compound comprises para-xylene.

23. The method of claim 19 further comprising, adding molecular oxygen to said CTA particles after said oxidizing of step (a).

24. The method of claim 19 further comprising, adding vapors comprising acetic acid to said digestion reactor to thereby provide heating in said digestion reactor.

25. The method of claim 19 wherein said crude slurry comprises agglomerations of said CTA particles, wherein said agglomerations have a mean particles size in the range of from about 50 to about 150 microns.

26. The method of claim 25 wherein the mean particle size of said PTA particles is in the range of from about 30 to about 100 microns.

27. An apparatus comprising: a primary oxidation vessel; a digestion vessel in fluid communication with said primary oxidation vessel; and a mechanical stirrer at least partly disposed in said digestion vessel and having less than five impellers, wherein said mechanical stirrer is configured to consume power at a rate in the range of from about 0.05 to about 1.5 kw per cubic meter of volume defined within said digestion vessel.

28. The apparatus of claim 27 wherein said mechanical stirrer is configured to consume power at a rate in the range of from about 0.1 to about 0.9 kw per cubic meter of volume defined within said digestion vessel.

29. The apparatus of claim 27 wherein said mechanical stirrer comprises an upright shaft and two to four vertically-spaced impellers coupled to said shaft.

30. The apparatus of claim 27 further comprising, an oxygen inlet for introducing additional molecular oxygen downstream of said primary oxidation vessel.
